(19) United States
(12) Patent Application Publication
(10) Pub. No.: US 2024/0412817 A1
(43) Pub. Date: Dec. 12, 2024

(54) IGK GENE REARRANGEMENT DETECTION METHOD AND APPARATUS, ELECTRONIC DEVICE, AND STORAGE MEDIUM

(71) Applicant: BOE Technology Group Co., Ltd., Beijing (CN)

(72) Inventor: Dan YUAN, Beijing (CN)

(21) Appl. No.: 18/701,259

(22) PCT Filed: May 16, 2023
(86) PCT No.: PCT/CN2023/094568

§ 371 Date: Apr. 15, 2024

(30) Foreign Application Priority Data
May 18, 2022 (CN) 202210552015.3

(51) Int. Cl. G16B 20/50; G16B 30/10; G16B 30/20; G16B 40/30

(52) U.S. Cl. CPC G16B 20/50; G16B 30/10; G16B 30/20; G16B 40/30

(57) ABSTRACT

An IGK gene rearrangement detection method and apparatus, an electronic device, and a storage medium. The detection method comprises: obtaining a first-end sequencing sequence and a second-end sequencing sequence of a test sample; assembling on the basis of the first-end sequencing sequence and the second-end sequencing sequence to obtain an assembled sequence; determining a target comparison gene from a gene reference database on the basis of the assembled sequence, the gene reference database comprising an IGKV gene library, an IGKJ gene library, a Kde gene library, and a J_C_intron gene library, and the target comparison gene comprising at least one of a target V gene, a target J gene, a target Kde gene, and a target J_C_intron gene; and determining an IGK gene rearrangement result in the assembled sequence on the basis of the target comparison gene.

### CROSS-REFERENCE TO RELATED APPLICATIONS

The present application is a U.S. National Phase Entry of International PCT Application No. PCT/CN2023/094568 having an international filing date of May 16, 2023, which claims the priority of the Chinese patent application No. 202210552015.3, entitled “IGK Gene Rearrangement Detection Method and Apparatus, Electronic Device, and Storage Medium”, filed to the CNIPA on May 18, 2022. The contents of the above-identified applications are incorporated herein by reference in their entireties.

### TECHNICAL FIELD

Embodiments of the disclosure relate to, but are not limited to, the technical field of gene detection, in particular to a detection method and an apparatus, an electronic device, and a storage medium for IGK gene rearrangement.

### BACKGROUND

Gene rearrangement occurs when pluripotent hematopoietic stem cells directionally differentiate into lymphocyte lines, and gene rearrangement sequences of each lymphocyte are unique, that is to say, genes of normal lymphocytes are polyclonal rearrangements. However, lymphoma cells and progeny cells thereof are from the same clone, and have the same encoding genes. Amplified products of deoxyribonucleic acid (DNA) from tumor cells show a specific band in a specific region upon electrophoresis. However, electrophoresis of amplified products of lymphocytes in patients with reactive hyperplasia of lymph nodes and normal people showed diffuse bands. At present, studies have shown that gene rearrangements are all acquired gene damages, and lymphoma cells are formed by monoclonal proliferation of cells with gene abnormalities, thus monoclonal changes occur. Such monoclonal gene rearrangements can be used as a specific molecular marker for detecting cell lymphoma in the diagnosis of B-cell lymphoma, and detection of such clonality is helpful to distinguish polyclonal reactive hyperplasia from malignant proliferative diseases.

Studies have shown that immunoglobulin kappa locus (IGK) gene rearrangement was found in 60% of childhood B-cell acute lymphoblastic leukemia (B-ALL) cases, and IGK gene rearrangement was related to the deletion rearrangement of Kappa-deleting element (Kde gene). The recombinant signal sequence of Kde gene is approximately located about 24 Kb downstream of the Constant(C) gene fragment, and the types of Kde rearrangement comprise 1) V-Kde rearrangement: the recombinant signal sequence of Kde can be rearranged into Variable (V) gene fragment, which leads to the deletion of Joining (J) gene and C gene; 2) J_C_intron-Kde rearrangement: the recombinant signal sequence in intron between J gene and C gene is rearranged with the recombinant signal sequence of Kde gene, which leads to the deletion of C gene.

### SUMMARY

Embodiments of the disclosure provide a detection method, an apparatus, an electronic device and a storage medium for IGK gene rearrangement.

In a first aspect, the embodiments of the present disclosure provide a detection method for IGK gene rearrangement, comprising:

obtaining paired-end sequencing data of a test sample; the paired-end sequencing data comprising a first-end sequencing sequence and a second-end sequencing sequence;

assembling based on the first-end sequencing sequence and the second-end sequencing sequence to obtain an assembled sequence;

determining a target alignment gene from a gene reference database based on the assembled sequence; wherein the gene reference database comprises an IGKV gene library, an IGKJ gene library, a Kde gene library and a J_C_intron gene library in a germ cell line, and the target alignment gene comprises at least one of a target V gene, a target J gene, a target Kde gene and a target J_C_intron gene;

determining an IGK gene rearrangement result in the assembled sequence based on the target alignment gene.

In some alternative embodiments, the first-end sequencing sequence comprises a plurality of first read sequences and the second-end sequencing sequence comprises a plurality of second read sequences;

assembling based on the first-end sequencing sequence and the second-end sequencing sequence to obtain an assembled sequence comprises:

traversing the first read sequence to determine a first similar read sequence corresponding to the first read sequence; taking a majority voting based on each group of the first read sequence and the first similar read sequence to obtain a first-end corrected sequence; and traversing the second read sequence to determine a second similar read sequence corresponding to the second read sequence; taking a majority voting based on each group of the second read sequence and the second similar read sequence to obtain a second-end corrected sequence;

assembling based on the first-end corrected sequence and the second-end corrected sequence to obtain the assembled sequence.

In some alternative embodiments, taking a majority voting based on each group of the first read sequence and the first similar read sequence to obtain a first-end corrected sequence comprises:

determining an amount of similarity based on each group of the first read sequence and the first similar read sequence; when the amount of similarity is greater than a set value, taking a majority voting on the bases at each position of the first read sequence and the first similar read sequence to obtain the first corrected read sequence; obtaining the first-end corrected sequence according to all the first corrected read sequences;

taking a majority voting based on each group of the second read sequence and the second similar read sequence to obtain a second-end corrected sequence comprises:

determining an amount of similarity based on each group of the second read sequence and the second similar read sequence; when the amount of similarity is greater than the set value, taking a majority voting on the bases at each position of the second read sequence and the second similar read sequence to obtain a second corrected read sequence; obtaining the second-end corrected sequence according to all the second corrected read sequences.

In some alternative embodiments, after obtaining the first-end corrected sequence and the second-end corrected sequence, the detection method further comprises:

trimming an adapter sequence from the first corrected read sequence to obtain a first preprocessed read sequence, and obtaining a first-end preprocessed sequence according to all the first preprocessed read sequences; and trimming an adapter sequence from the second corrected read sequence to obtain a second preprocessed read sequence, and obtaining a second-end preprocessed sequence according to all the second preprocessed read sequences;

assembling based on the first-end corrected sequence and the second-end corrected sequence to obtain the assembled sequence comprises:

assembling based on the first-end preprocessed sequence and the second-end preprocessed sequence to obtain the assembled sequence.

In some alternative embodiments, after obtaining the first-end preprocessed sequence and the second-end preprocessed sequence, the detection method further comprising:

deleting a first preprocessed read sequence having a length lower than a first set length to obtain a first-end sequence to be assembled; and deleting a second preprocessed read sequence having a length lower than the first set length to obtain a second-end sequence to be assembled;

assembling based on the first-end preprocessed sequence and the second-end preprocessed sequence to obtain the assembled sequence comprises:

assembling based on the first-end sequence to be assembled and the second-end sequence to be assembled to obtain the assembled sequence.

In some alternative embodiments, a value of the first set length ranges from 10 bp to 100 bp.

In some alternative embodiments, assembling based on the first-end sequence to be assembled and the second-end sequence to be assembled to obtain the assembled sequence comprises:

obtaining a reverse complementary read sequence of the second preprocessed read sequence;

determining an overlapping sequence according to the first preprocessed read sequence and the reverse complementary read sequence;

when a length of the overlapping sequence is not lower than a second set length, deleting the overlapping sequence from the reverse complementary read sequence to obtain the read sequence to be assembled;

splicing the first preprocessed read sequence and the read sequence to be assembled to obtain an assembled read sequence;

obtaining the assembled sequence based on all the assembled read sequences.

In some alternative embodiments, determining a target alignment gene from a target gene reference database based on the assembled sequence comprises:

determining a target alignment gene corresponding to each of the assembled read sequences from the target gene reference database based on set alignment parameters.

In some alternative embodiments, the set alignment parameter comprises: the similarity between alignment fragments in the assembled read sequence and the target alignment gene being not less than 90%, and a length of the alignment fragment ranging from 4 to 11.

In some alternative embodiments, when the target alignment gene comprises only the target V gene and the target J gene, determining an IGK gene rearrangement result in the assembled sequence based on the target alignment gene comprises:

obtaining a nucleotide position comprising a phenylalanine residue in the target J gene, and determining a termination point in the assembled sequence based on the nucleotide position;

detecting a cysteine residue in the assembled sequence within a set range before the termination point, and taking a position of the cysteine residue closest to the termination point as a starting point; the set range being assembled sequence fragments from the termination point to 60 bp to 90 bp before the termination point;

determining a CDR3 region in the assembled sequence according to the starting point and the termination point.

In some alternative embodiments, when the target alignment gene comprises only the target V gene and the target J gene, determining an IGK gene rearrangement result in the assembled sequence based on the target alignment gene comprises:

performing clustering analysis on the assembled sequence based on the target V gene and the target J gene to obtain a quantity of clone sequences and a proportion of clone sequences in the assembled sequence.

In a second aspect, the embodiments of the present disclosure provide a detection apparatus for IGK gene rearrangement, comprising:

an acquisition module configured to obtain paired-end sequencing data of a test sample; the paired-end sequencing data comprising a first-end sequencing sequence and a second-end sequencing sequence;

an assembly module configured to assemble based on the first-end sequencing sequence and the second-end sequencing sequence to obtain an assembled sequence;

an alignment module configured to determine a target alignment gene from a gene reference database based on the assembled sequence; wherein the gene reference database comprises an IGKV gene library, an IGKJ gene library, a Kde gene library and a J_C_intron gene library in a germ cell line, and the target alignment gene comprises at least one of a target V gene, a target J gene, a target Kde gene and a target J_C_intron gene;

a determination module configured to determine an IGK gene rearrangement result in the assembled sequence based on the target alignment gene.

In a third aspect, embodiments of the present disclosure provide an electronic device comprising a processor and a memory coupled to the processor, the memory storing instructions that, when executed by the processor, cause the electronic device to perform steps of the detection method of any of the embodiments of the first aspect.

In a fourth aspect, embodiments of the present disclosure provide a computer-readable storage medium having stored thereon a computer program, wherein when the computer program is executed by a processor, steps of the detection method of any of the embodiments of the first aspect are implemented.

Other aspects of the present disclosure may be comprehended after the drawings and the detailed descriptions are read and understood.

### BRIEF DESCRIPTION OF DRAWINGS

FIG. 1 shows a flow schematic diagram of a detection method provided by an embodiment of a first aspect of the present disclosure;

FIG. 2 shows a length distribution schematic diagram of an assembled read sequence provided by an embodiment of the first aspect of the present disclosure;

FIG. 3 shows a schematic diagram of a detection apparatus provided by an embodiment of a second aspect of the present disclosure;

FIG. 4 shows a schematic diagram of an electronic device provided by an embodiment of a third aspect of the present disclosure; and

FIG. 5 shows a schematic diagram of a computer-readable storage medium provided by an embodiment of a fourth aspect of the present disclosure.

### DETAILED DESCRIPTION

Exemplary embodiments of the present disclosure will be described below in more detail with reference to accompanying drawings. Although the exemplary embodiments of the present disclosure are shown in the accompanying drawings, it should be understood that the present disclosure may be implemented in various forms and should not be limited by the embodiments set forth herein. On the contrary, these embodiments are provided for a more thorough understanding of the present disclosure and for fully conveying the scope of the present disclosure to a person skilled in the art.

Gene rearrangement detection tools (including MiGEC, Mixcr, IgBlast, etc.) are all used to identify variable-(diversity)-joining (V(D)J) gene rearrangement of genes, such as Immunoglobulin Heavy chain (IGH), IGK, T cell Receptor Beta locus (TRB), and T cell Receptor Delta locus (TRD), but there is a lack of schemes for detection of Variable-Joining (VJ) gene rearrangement of IGK genes, V-Kde gene rearrangement and J_C_intron-Kde gene rearrangement.

In order to detect VJ gene rearrangement, V-Kde gene rearrangement and J_C_intron-Kde gene rearrangement in IGK gene rearrangement, an embodiment of the present disclosure provides a detection method for IGK gene rearrangement, comprising:

obtaining paired-end sequencing data of a test sample; the paried-end sequencing data comprising a first-end sequencing sequence and a second-end sequencing sequence; obtaining an assembled sequence by assembling based on the first-end sequencing sequence and the second-end sequencing sequence; determining a target alignment gene from gene reference database based on the assembled sequence; wherein the gene reference database comprises an Immunoglobulin Kappa Variable (IGKV) gene library, an Immunoglobulin Kappa Joining (IGKJ) gene library, a Kde gene library and a J_C_intron gene library in a germ cell line, and the target alignment gene comprises at least one of a target V gene, a target J gene, a target Kde gene and a target J_C_intron gene; determining IGK gene rearrangement results in the assembled sequence based on the target alignment gene.

The above scheme obtains the assembled sequence based on assembling the first-end sequencing sequence and the second-end sequencing sequence in the raw data of the paired-end sequencing, and aligns the assembled sequence with gene reference sequences in the IGKV gene library, the IGKJ gene library, the Kde gene library and the J_C_intron gene library of the germ cell line, and determines a target alignment gene from the gene library, comprising at least one of the target V gene, the target J gene, the target Kde gene and the target J_C_intron gene; determines the IGK gene rearrangement result in the assembled sequence based on the target alignment gene. The above method provides a scheme for automatic flow detection of VJ gene rearrangement, V-Kde gene rearrangement and J_C_intron-Kde gene rearrangement in IGK genes, which is suitable for downstream analysis and identification of requirements, such as minimal residual disease (MRD) and relapse monitoring of lymphoma, immune repertoire sequencing.

In the following contents, further description is made in connection with exemplary embodiments.

Explanations of some key English terms involved in the exemplary embodiments:

B-cell Receptor (BCR): an Immunoglobulin molecule (IG) growing on the surface of B lymphocytes, which consists of two heavy chains (IGH) and two light chains (IGK or Immunoglobulin lambda locus (IGL)).

IGK light chain: consisting of arrangement of a constant region (C gene) and a variable region (V gene, J gene).

Human IGK gene: located on the short arm of chromosome 2 (2p11.2), comprising C genes, Kde genes, various V genes and J genes.

Complementarity-determining Region 3 (CDR3 region): a region of the variable region that determines the object to be recognized, comprising the end of V gene and the front end of J gene.

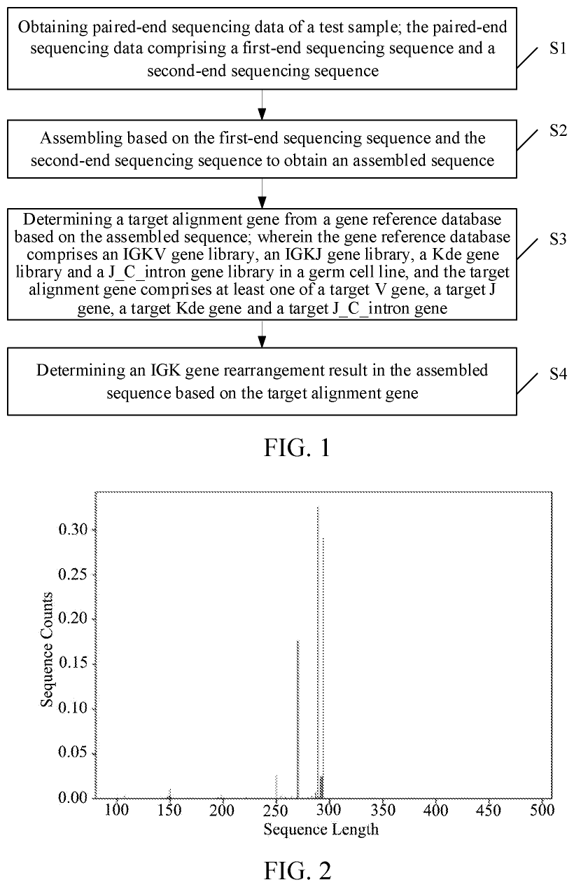

In an exemplary embodiment, a detection method for IGK gene rearrangement based on a high-throughput sequencing technology or a “next-generation” sequencing technology (NGS) is provided, with reference to FIG. 1, comprising steps S1 to S4 as follows:

S1: obtaining paired-end sequencing data of a test sample; the paired-end sequencing data comprising a first-end sequencing sequence and a second-end sequencing sequence.

In an exemplary embodiment, the test sample is a sample to be tested of lymphocytes, and after nucleic acid extraction, library construction and other steps, the test sample is sent to a high-throughput sequencer for sequencing to obtain the paired-end sequencing data.

The paired-end sequencing refers to sequencing of a deoxyribonucleic acid (DNA) strand in both forward and reverse directions. In the embodiment, the first-end sequencing sequence represents a nucleic acid sequence obtained by sequencing in a first direction of DNA in the paired-end sequencing, and the second-end sequencing sequence represents a nucleic acid sequence obtained by sequencing in a second direction of DNA in the paried-end sequencing. The first direction is opposite to the second direction. For example, the first direction may be a direction from left to right and the second direction may be a direction from right to left.

Taking a commonly used characterization standards of high-throughput sequencing data as an example, information of the first-end sequencing sequence and the second-end sequencing sequence are stored in a fastq file, respectively, which is mainly used to save base sequences and sequencing quality. The base sequence and sequencing quality are indicated by American Standard Code for Information Interchange (ASCII) code.

A plurality of reads are stored in one fastq file; a read is a read sequence, also known as a sequencing short sequence, which is a base sequence obtained by a single sequencing via a high-throughput sequencer. One read in the fastq file has four lines of information, an example of which is as follows:

@SRR835775.1 1/1
TAACCCTAACCCTAACCCTAACCCTA . . .
+
???B1ADDD8??BB+C?B+:AA883CEE . . .

wherein, the first line is the sequence number id and description information of the read, beginning with @; the second line is the base sequence; the third line begins with a plus sign, which is a sequence indication and description; the fourth line is quality information corresponding to the base sequence in the second line.

Therefore, the first-end sequencing sequence comprises a plurality of first read sequences and the second-end sequencing sequence comprises a plurality of second read sequences.

For ease of description and distinction, in embodiments of the present disclosure, the first-end sequencing sequence and the sequences obtained after subsequent processing thereof are uniformly labeled as Read1, abbreviated as R1, and the second-end sequencing sequence and the sequences obtained after subsequent processing thereof are uniformly labeled as Read2, abbreviated as R2; the reads in R1 are marked as r₁ᵢ and the reads in R2 as r₂ᵢ; 1≤i≤N and being an integer; wherein, i is a quantity of reads, and N is a quantity of reads comprised in the first-end sequencing sequence or the second-end sequencing sequence.

S2: obtaining an assembled sequence by assembling based on the first-end sequencing sequence and the second-end sequencing sequence.

assembling a sequence is assembling or splicing the first read sequence in the first-end sequencing sequence and the second read sequence in the second-end sequencing sequence according to the corresponding relationship of gene sequencing or the sequence number id of reads, thus obtaining a complete and assembled sequence. Existing tools, such as Pear or Pandaseq, without limitation here, can be invoked when assembling.

In some alternative embodiments, prior to assembling, the first-end sequencing sequence and the second-end sequencing sequence may be subjected to data quality check and preprocessed, with the aim of removing low-quality reads to obtain high-quality data for assembly, thereby improving the accuracy of the subsequent target gene alignment.

An alternative scheme in quality check and preprocessing is to correct the paired-end sequencing data, comprising the following operations:

after obtaining the paired-end sequencing data of the test sample, traversing the first read sequence to determine a first similar read sequence corresponding to the first read sequence; taking a majority voting based on each group of the first read sequence and the first similar read sequence, to obtain a first-end corrected sequence; traversing the second read sequence to determine a second similar read sequence corresponding to the second read sequence; taking a majority voting based on each group of the second read sequence and the second similar read sequence, to obtain a second-end corrected sequence.

Exemplarily, similarities between each reads and other reads in the first-end sequencing sequence may be calculated, respectively, and other reads having similarity greater than a set threshold is regarded as the similar read sequence of the reads.

For example, as for the first read sequence r₁₁ in R1, similarities between r₁₁ and r₁₂, r13, . . . , r_{1N} are calculated sequentially, then r₁ⱼ having similarity greater than the set threshold is regarded as the similar read sequence of r₁₁. In the same way, the corresponding similar read sequences of r₁₂, r₁₃, . . . , r_{1N} are determined sequentially. The method for calculating similarity between base sequences may be realized by using the prior art and will not be described here. The set threshold is determined according to requirements, which is not limited here.

Next, take a majority voting based on each group of the first read sequence and the first similar read sequence corresponding to the group of the first read sequence. The majority voting is a corrective scheme in which as for an array containing n elements, majority elements therein are found and minority elements are replaced by the majority elements; the majority elements may, for example, refer to the element that appears more than [n/2] times in the array. The first-end sequencing sequence is corrected by the majority voting, and the first-end corrected sequence is obtained, so as to reduce sequencing errors in the process of gene sequencing and improve the accuracy of the subsequent gene alignment and analysis.

Alternatively, each base in reads is used as a voting element. For each group of the first read sequence r₁ᵢ and the first similar read sequences r₁ⱼ corresponding to r₁ᵢ, bases at the same position are sequentially taken out for the majority voting, and then the bases determined by the majority voting are used as corrected bases at the position.

In an exemplary embodiment, taking the first-end sequencing sequence as an example, an alternative method for taking a majority voting based on each group of the first read sequence and the first similar read sequence, to obtain a first-end corrected sequence is as follows:

determining an amount of similarity based on each group of the first read sequence and the first similar read sequence; when the amount of similarity is greater than the set value, taking a majority voting on the bases at each position in the first read sequence and the first similar read sequence to obtain the first corrected read sequence; obtaining the first-end corrected sequence based on all the first corrected read sequences.

In an exemplary embodiment, the amount of similarity is counted when internal similarity calculation is performed on the first-end sequencing sequence, and the amount of similarity of the first read sequence is automatically added 1 when a similar read sequence is found for any first read sequence. For example, as for the read sequence r₁₁, when reads found similar to r₁₁ via the similarity calculation are: r₁₂ and r₁₅, the amount of similarity is 2; as for the read sequence r₁₂, when reads found similar to r₁₂ via the similarity calculation are: r₁₃, r₁₄ and r₁₇, the amount of similarity is 3.

After counting each group of the first read sequence and the corresponding first similar read sequence, take a majority voting on the group of the first read sequence and the first similar read sequence having the amount of similarity greater than the set value to obtain the corresponding first corrected read sequence; the group of the first read sequence and the first similar read sequence having the amount of similarity less than or equal to the set value may be directly deleted, and will not participate in subsequent sequence assembly and alignment. The set value may be from 1 to 3, for example preferably 2, i.e. the majority voting taken only on the first read sequence and the first similar read sequence having the amount of similarity greater than 2.

For example, as for a group of the first read sequence and the first similar read sequence, the total quantity of reads is 5>2, then a majority voting is taken on the five reads. If the bases at the first position of the five reads are A, T, A, A and T, respectively, majority bases are determined as A according to the principle of the majority voting, and the bases at the first position of the first read sequence or all the five reads are uniformly corrected as A. Then, according to the same method, a majority voting is taken on the bases at the second position, the bases at the third position, until the bases at the last position of the five reads, respectively, and the corrected first read sequence is determined as the first corrected read sequence.

The first-end corrected sequence may be obtained after the majority voting correction of all groups of the first read sequence and the first similar read sequence is completed.

The same as the first-end sequencing sequence is for the second-end sequencing sequence, which is as follows:

determining an amount of similarity based on each group of the second read sequence and the second similar read sequence; when the amount of similarity is greater than the set value, taking a majority voting on the bases at each position in the second read sequence and the second similar read sequence to obtain the second corrected read sequence; obtaining the second-end corrected sequence based on all the second corrected read sequences.

The above method can correct amplification errors in the process of gene sequencing by performing similarity calculation within the first-end sequencing sequence and the second-end sequencing sequence, and retaining reads having the amount of similarity greater than the set value for the majority voting, so as to improve the reliability of sequencing sequences and improve the accuracy of the subsequent target gene alignment.

In some alternative embodiments, before correcting the sequencing sequences via the majority voting method, reads containing unknown nucleotides, i.e. containing base N, in the first-end sequencing sequence and the second-end sequencing sequence may also be removed, and reads having an average base quality lower than a set quality may be removed to further improve the data quality of the sequencing sequences and reduce the workload of correcting the sequencing sequences. The value range of the set quality may be 20 to 25, for example, 20.

In some alternative embodiments, after completion of the majority voting correction of the sequencing sequences, the detection method further comprises:

trimming an adapter sequence from the first corrected read sequence to obtain a first preprocessed read sequence, and obtaining a first-end preprocessed sequence according to all the first preprocessed read sequences; and trimming an adapter sequence from the second corrected read sequence to obtain a second preprocessed read sequence, and obtaining a second-end preprocessed sequence according to all the second preprocessed read sequences; after trimming the adapter sequences, the first-end preprocessed sequence and the second-end preprocessed sequence may be used to enter the subsequent assembly step.

The adapter sequence (adaptor) is a known short sequence added at both ends of the target sequencing fragment in the process of high-throughput sequencing, which is used to distinguish different test samples in mixed sequencing. Therefore, the adapter sequence can be trimmed before assembling.

Taking the first-end corrected sequence as an example, the adapter sequence may be trimmed using the following methods:

taking the first 4000 to 10000 lines of R1, and retrieving to identify and filter the adapter sequences added by different sequencing platforms; when detecting that the left and right ends of a certain r₁ᵢ and the adapter sequence overlaps in a length greater than or equal to 3 bp, the portion of the fragment is determined as the adapter sequence and trimmed.

In some alternative embodiments, after obtaining the first-end preprocessed sequence and the second-end preprocessed sequence and before assembling, the detection method further comprises:

deleting a first preprocessed read sequence having a length lower than the first set length to obtain a first-end sequence to be assembled; and deleting a second preprocessed read sequence having a length lower than the first set length to obtain the second-end sequence to be assembled.

In an exemplary embodiment, after trimming the adapter sequence, all reads having a length less than the first set length are removed based on the lengths of the reads in R1 and R2. When a length of a reads in R1: r₁ₐ is lower than the first set length, r₁ₐ in R1 and r_{2b} corresponding to r₁ₐ in R2 are deleted synchronously. The first set length (trim_len) represents a parameter of length of preprocessed single-end reads, which can be adjusted according to actual needs, and the adjustable range is 10 bp to 100 bp, and bp is one base pair.

By deleting the reads having single-end length less than the first set length in the first-end preprocessed sequence and the second-end preprocessed sequence before assembling, the sequencing fragments unrelated to V gene, J gene, Kde gene and J_C_intron gene in the sequencing sequence can be reduced, thereby reducing the interference of invalid gene fragments and non-target alignment gene fragments during the subsequent gene library alignment, and reducing the workload of gene alignment and improving the accuracy of gene alignment.

Next, assembly is performed based on the first-end sequence to be assembled and the second-end sequence to be assembled to obtain an assembled sequence.

An alternative assembly scheme is as follows:

obtaining a reverse complementary read sequence of the second preprocessed read sequence; determining an overlapping sequence according to the first preprocessed read sequence and the reverse complementary read sequence; deleting the overlapping sequence from the reverse complementary read sequence when the length of the overlapping sequence is not lower than a second set length to obtain the read sequence to be assembled; splicing the first preprocessed read sequence and the read sequence to be assembled to obtain an assembled read sequence; obtaining the assembled sequence based on all the assembled read sequences.

In an exemplary embodiment, all reads: r₂ᵢ in R2 are transformed into reverse complementary reads thereof, marked as r₂ᵢ′, and then r₁ᵢ is aligned with r₂ᵢ′ corresponding to r₁ᵢ, to determine an overlapping sequence between them and determine the overlapping sequence length overlap. When overlap≥overlap_len, the overlapping sequence is removed from r₂ᵢ′, and then r₁ᵢ and the remaining sequence in r₂ᵢ are connected to get an assembled read sequence (assembled) and marked as query_id. Wherein, the overlap_len is the second set length, that is, the minimum overlapping sequence length, with a value range from 10 bp to 40 bp.

When the length of the overlapping sequence between r₁ᵢ and r₂ᵢ′ overlap<overlap_len, then such group of r₁ᵢ and r₂ᵢ′ are saved as assembly failure sequences (assembled_F and assembled_R) respectively, and the assembly failure sequences do not participate in the subsequent gene alignment.

The assembled sequence is obtained by splicing all the first preprocessed read sequences with the read sequences to be assembled, and the assembled sequence comprises a plurality of assembled read sequences query_id.

S3: determining a target alignment gene from the gene reference database based on the assembled sequence; wherein the gene reference database comprises an IGKV gene library, an IGKJ gene library, a Kde gene library and a J_C_intron gene library in a germ cell line, and the target alignment gene comprises at least one of a target V gene, a target J gene, a target Kde gene and a target J_C_intron gene.

In an exemplary embodiment, in order to analyze the VJ gene rearrangement, V-Kde gene rearrangement and J_C_intron-Kde gene rearrangement in the IGK gene, each query_id sequence is locally aligned with the V/J/Kde/J_C_intron gene reference sequence of the germline sequentially to determine from which V/J/Kde/J_C_intron gene was the assembled sequence recombined, thereby determining the gene rearrangement of the assembled sequence.

An alternative alignment scheme is as follows:

as for VJ gene rearrangement:

sequentially aligning each query_id with a plurality of V and J gene sequences of IMGT immune repertoire data IGK, and finding the target alignment gene meeting the set alignment parameters, and recording id of the extracted gene as subject_id.

as for V-Kde gene rearrangement and J_C_intron-Kde gene rearrangement:

sequentially aligning each query_id sequence with J_C_intron library and Kde gene library of IGK, and finding the target alignment gene meeting the set alignment parameters, and recording id of the extracted gene as subject_id.

Alternatively, the set alignment parameters comprise: the similarity between the alignment fragment in the assembled read sequence and the target alignment gene being not less than 90%, and the length of the alignment fragment ranging from 4 to 11. The above alignment parameters can improve the speed and accuracy of obtaining target alignment genes, namely V gene, J gene, Kde gene and J_C_intron gene, from gene reference database.

During implementation, the alignment can be carried out in IGKV, IGKJ, J-C_intron and Kde gene reference databases by using the blastn tool and inputting the set alignment parameters. When the alignment is successful, the id of the target alignment gene is extracted. The set alignment parameters in the blastn tool are: 1) the similarity parameter between the alignment fragment and the target alignment gene: -perc_identity=90; 2) the length of the sequence fragment -word_size=4 to 11, for example, 11.

By alignment using the above set alignment parameters, the best target alignment gene among 114 IGKV genes, 9 IGKJ genes, Kde genes and J_C_intron genes can be found.

S4: determining the IGK gene rearrangement result in the assembled sequence based on the target alignment gene.

After being obtained from the gene reference database, the target alignment gene can be used to annotate the sequence fragments in the assembled sequence, so as to obtain the rearrangement result or rearrangement state of IGK gene, which can be used for subsequent identification and analysis of IGK gene rearrangement.

When VJ gene rearrangement in IGK gene is detected, identification of CDR3 sequence can be performed. One approach is to define the CDR3 region as a sequence fragment from the second conserved cysteine residue at the 3′ end of the V gene to the conserved phenylalanine residue in the J gene. However, studies have shown that the second conserved cysteine residue may not be the last cysteine on the V gene, so the disclosed embodiment provides a more accurate scheme to determine the starting position of the CDR3 region.

In some alternative embodiments, when the target alignment gene subject_id obtained according to the query_id sequence alignment contains only the V gene and the J gene, determining the IGK gene rearrangement result in the assembled sequence based on the target alignment gene further comprises annotating the CDR3 region thereof, for example comprising:

obtaining a nucleotide position comprising a phenylalanine residue in the target J gene, and determining a termination point in the assembled sequence based on the nucleotide position; detecting the cysteine residues in the assembled sequence within the set range before the termination point, and taking the position of the cysteine residue closest to the termination point as a starting point; the set range being assembled sequence fragments from the termination point to 60 bp to 90 bp before the termination point; determining the CDR3 region in the assembled sequence according to the starting point and termination point.

In an exemplary embodiment, according to the target J gene obtained by alignment, the nucleotide position corresponding to the phenylalanine residue in “FGXG” of the target J gene is detected, and the termination point of the CDR3 region in the assembled sequence is determined; then, the cysteine residues were searched in the length range from 60 bp to 90 bp before the CDR3 termination point, and the cysteine residue found to be closest to the termination point was taken as the starting point of CDR3 region, so that the CDR3 sequence was determined according to the starting point and the termination point. An alternative set range is an assembled sequence fragments within the length range of 75 bp before the termination point.

In the J gene obtained according to the alignment in the above scheme, the nucleotide position corresponding to phenylalanine residue in “FGXG” is detected to determine the position of the termination point of the CDR3 region in the sequence, and then the cysteine residue is searched in the range of 60 bp to 90 bp before the position, and the last cysteine residue is used as the starting point of the CDR3 region. Searching for the closest cysteine residue in the search range from 60 bp to 90 bp can ensure that the cysteine residue found is the last cysteine residue before phenylalanine residue, thus improving the determination accuracy of CDR3 region.

In addition, the current gene rearrangement detection tools do not carry out relevant immune repertoire function analysis on VJ gene rearrangement results in IGK, such as clone diversity and analysis of common clone among multiple samples. Therefore, the embodiments of the present disclosure also provide an automatic detection and analysis scheme for VJ gene rearrangement identification and immune repertoire analysis for IGK.

In some alternative embodiments, when the target alignment gene subject_id obtained from the query_id sequence alignment contains only the V gene and the J gene, determining the IGK gene rearrangement result in the assembled sequence based on the target alignment gene further comprises cloning analysis thereof, for example comprising:

performing cluster analysis on the assembled sequences based on target V gene and target J gene to obtain a quantity of clone species, a quantity of clone sequences and a proportion of clone sequences in the assembled sequences. After obtaining the data of clone species, the quantity of the clone sequences and the proportion of the clone sequences, analysis of common clones can be performed, to dig deep into relationships between immune repertoire and diseases.

In order to more intuitively illustrate the results of clone analysis of VJ gene rearrangement, in an alternative embodiment, illustrations are made in conjunction with exemplary embodiments:

after obtaining the raw data of paired-end sequencing of a test sample, the following was performed sequentially: removing the reads comprising unknown nucleotides (N), removing the reads with average base quality less than 20, taking a majority voting correction on the reads with the amount of similarity >2, trimming the adapter sequence and then assembling to obtain the assembled sequence.

First, the statistical visualization analysis is carried out according to the length of the assembled read sequence, with reference to a length distribution schematic diagram of the assembled read sequence provided in FIG. 2. In FIG. 2, sequence counts as the ordinate represents a proportion of the quantity of assembled read sequences; and sequence length as the abscissa represents a sequence length in bp of the assembled read sequence. As can be seen from FIG. 2, there are a plurality of peaks of the quantity in the whole assembled sequence, indicating that there may be polyclonal types in the assembled sequence.

When carrying out gene alignment using each assembled read sequence, exemplary results of gene alignment with IGKV, IGKJ, J_C_intron, and Kde gene reference databases are shown in Table 1:

TABLE 1
Alignment results of an assembled read sequence

| | | % | alignment | | | q. | q. | s. | s. | |
|---|---|---|---|---|---|---|---|---|---|---|
| query_id | Subject_id | identity | length | mismatches | gaps | start | end | start | end | evalue |
| rₐ | IGKV2D-40*0.1 | 100 | 227 | 0 | 0 | 45 | 271 | 303 | 77 | 2.39E−120 |
| rₐ | IGKV2-40*0.1 | 100 | 227 | 0 | 0 | 45 | 271 | 303 | 77 | 2.39E−120 |
| rₐ | IGKJ4*0.1 | 100 | 37 | 0 | 0 | 6 | 42 | 38 | 2 | 8.27E−17 |
| rₐ | J5-C-intron | 100 | 13 | 0 | 0 | 100 | 112 | 2405 | 2417 | 0.028 |

According to the target alignment gene obtained by alignment, namely Subject_id gene, clone analysis of VJ rearrangement is carried out on all assembled read sequences, and a quantity of clone types, a quantity of sequences supporting the clone and a proportion of sequences supporting the clone are calculated, as shown in Table 2:

TABLE 2
Clone analysis of VJ rearrangement

| Clone Number/top 10 | Sequence count | Proportion of Sequence/% |
|---|---|---|
| 1 | 247438 | 47.6 |
| 2 | 222903 | 42.9 |
| 3 | 9194 | 1.8 |
| 4 | 8730 | 1.7 |
| 5 | 2625 | 0.5 |
| 6 | 2144 | 0.4 |
| 7 | 2224 | 0.4 |
| 8 | 1915 | 0.4 |
| 9 | 1402 | 0.3 |
| 10 | 1392 | 0.3 |

It can be seen from the top 10 clones that the quantities of sequences (count) of the first clone and the second clone are comparative, accounting for more than 40% of the assembled sequences. It can be seen that the IGK rearrangement of the current test samples belongs to a polyclonal type.

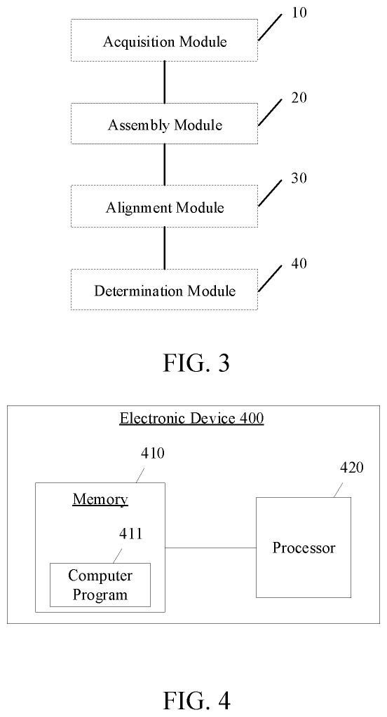

Embodiments of the present disclosure also provide a detection apparatus for IGK gene rearrangement. With reference to FIG. 3, the detection apparatus comprises:

an acquisition module 10 configured to obtain the paired-end sequencing data of a test sample; the paired-end sequencing data comprising a first-end sequencing sequence and a second-end sequencing sequence;

an assembly module 20 configured to assemble based on the first-end sequencing sequence and the second-end sequencing sequence to obtain an assembled sequence;

an alignment module 30 configured to determine a target alignment gene from a gene reference database based on the assembled sequence; wherein the gene reference database comprises an IGKV gene library, an IGKJ gene library, a Kde gene library and a J_C_intron gene library in a germ cell line, and the target alignment gene comprises at least one of a target V gene, a target J gene, a target Kde gene and a target J_C_intron gene;

a determination module 40 configured to determine the IGK gene rearrangement result in the assembled sequence based on the target alignment gene.

Alternatively, the first-end sequencing sequence comprises a plurality of first read sequences and the second-end sequencing sequence comprises a plurality of second read sequences;

The assembly module 20 is configured to:

traverse the first read sequence to determine a first similar read sequence corresponding to the first read sequence; take a majority voting based on each group of the first read sequences and the first similar read sequences to obtain a first-end corrected sequence; and traverse the second read sequence to determine a second similar read sequence corresponding to the second read sequence; take a majority voting based on each group of the second read sequences and the second similar read sequences to obtain a second-end corrected sequence;

assemble based on the first-end corrected sequence and the second-end corrected sequence to obtain an assembled sequence.

Alternatively, the assembly module 20 is configured to:

determine an amount of similarity based on each group of the first read sequence and the first similar read sequence; when the amount of similarity is greater than a set value, take a majority voting on the bases at each position in the first read sequence and the first similar read sequence to obtain a first corrected read sequence; obtain a first-end corrected sequence according to all the first corrected read sequences;

determine an amount of similarity based on each group of the second read sequences and the second similar read sequences; when the amount of similarity is greater than the set value, take a majority voting on the bases at each position in the second read sequence and the second similar read sequence to obtain a second corrected read sequence; obtain a second-end corrected sequence according to all the second corrected read sequences.

Alternatively, the assembly module 20 is configured to:

trim an adapter sequence from the first corrected read sequence to obtain a first preprocessed read sequence, and obtain a first-end preprocessed sequence according to all the first preprocessed read sequences; and trim an adapter sequence from the second corrected read sequence to obtain a second preprocessed read sequence, and obtain a second-end preprocessed sequence according to all the second preprocessed read sequences;

assemble based on the first-end preprocessed sequence and the second-end preprocessed sequence to obtain an assembled sequence.

Alternatively, the assembly module 20 is configured to:

delete the first preprocessed read sequence having a length lower than the first set length to obtain a first-end sequence to be assembled; and delete the second preprocessed read sequence having a length lower than the first set length to obtain the second-end sequence to be assembled;

assemble based on the first-end preprocessed sequence and the second-end preprocessed sequence to obtain an assembled sequence, comprising:

assemble based on the first-end sequence to be assembled and the second-end sequence to be assembled, and obtaining an assembled sequence.

In an exemplary embodiment, the assembly module 20 is configured to:

obtain a reverse complementary read sequence of the second preprocessed read sequence;

determine an overlapping sequence according to the first preprocessed read sequence and the reverse complementary read sequence;

when the length of the overlapping sequence is not lower than the second set length, delete the overlapping sequence from the reverse complementary read sequence to obtain the read sequence to be assembled;

splice the first preprocessed read sequence and the read sequence to be assembled to obtain an assembled read sequence;

obtain the assembled sequence based on all the assembled read sequences.

Alternatively, the alignment module 30 is configured to:

determine a target alignment gene corresponding to each assembled read sequence from the target gene reference database based on the set alignment parameters;

the set alignment parameters comprising: the similarity between the alignment fragment in the assembled reading sequence and the target alignment gene being not less than 90%, and the length of the alignment fragment ranging from 4 to 11.

Alternatively, when the target alignment gene comprises only the target V gene and the target J gene, the determination module 40 is configured to:

obtain a nucleotide position comprising a phenylalanine residue in the target J gene, and determine a termination point in the assembled sequence based on the nucleotide position;

detect cysteine residues in the assembled sequence within the set range before the termination point, and take the position of the cysteine residue closest to the termination point as the starting point; the set range is assembled sequence fragments from the termination point to 60 bp to 90 bp before the termination point;

determine a CDR3 region in the assembled sequence according to the starting point and termination point.

Alternatively, when the target alignment gene comprises only the target V gene and the target J gene, the determination module 40 is configured to:

perform cluster analysis on the assembled sequences based on target V gene and target J gene to obtain a quantity of clone sequences and a proportion of clone sequences in the assembled sequences.

Embodiments of the present disclosure also provide an electronic device 400, with reference to FIG. 4, comprising a processor 420 and a memory 410 coupled to the processor 420. The memory 410 stores a computer program 411 that, when executed by the processor 420, causes the electronic device 400 to perform the steps of the detection method described in the preceding embodiments.

An operating system as well as a third-party application program is installed in an electronic device, for example. Electronic devices can be servers, desktop computers, tablet computers, notebook computers, mobile phones, wearable devices, vehicle terminals and other electronic devices.

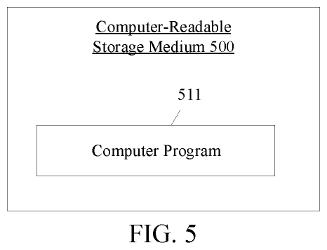

With reference to FIG. 5, the embodiments of the present disclosure also provide a computer-readable storage medium 500 having stored thereon a computer program 511. When the computer program 511 is executed by a processor, the steps of the detection method described in the preceding embodiments are implemented.

For brief description, where embodiments of devices, electronic devices and computer-readable storage media are not mentioned, reference may be made to the corresponding contents in the preceding detection method embodiments.

In general, the embodiments of the invention provide a method, a device, an electronic device and a storage medium for detecting IGK gene rearrangement, which: obtains an assembled sequence by assembling based on a first-end sequencing sequence and a second-end sequencing sequence in a paired-end sequencing raw data; aligns the assembled sequence with a gene reference sequence in an IGKV gene library, an IGKJ gene library, a Kde gene library and a J_C_intron gene library of a germ cell line, determines a target alignment gene from the gene library, including at least one of a target V gene, a target J gene, a target Kde gene and a target J_C_intron gene; determines an IGK gene rearrangement result in the assembled sequence based on a target alignment gene. The above method provides a scheme for automatic process detection of VJ gene rearrangement, V-Kde gene rearrangement and J_C_intron-Kde gene rearrangement in IGK gene, which is suitable for downstream analysis and identification of lymphoma minimal residual disease and recurrence monitoring, immune repertoire sequencing and other requirements.

It should be noted that, the term “and/or” in the present disclosure only describes a relationship of related objects, indicating that there may be three kinds of relationships, for example, A and/or B, may indicate: A alone, both A and B, and B alone. In addition, the character “/” in the present disclosure generally indicates that the object before “/” and the related object after “/” are in a relationship of “or”; the word “comprise” does not exclude the existence of elements or steps not listed in the claims. The word “a” or “an” before an element does not exclude the existence of a plurality of such elements. The invention can be implemented by means of hardware comprising several different elements and by means of a suitably programmed computer. In the unit claims enumerating a plurality of devices, several of these devices may be embodied by the same hardware item. The use of words first, second, third and so on does not indicate any order. These words can be interpreted as names.

It should be understood by those skilled in the art that the embodiments of the present disclosure may be provided as methods, systems, or computer program products. Therefore, for the present disclosure, a form of an entire hardware embodiment, an entire software embodiment, or an embodiment combining software and hardware aspects may be adopted. Furthermore, for the present disclosure, a form of a computer program product implemented on one or more computer usable memory media (including but not limited to a magnetic disk memory, a Compact Disc Read Only Memory (CD-ROM), and an optical memory, etc.) containing computer usable program codes therein may be adopted.

The present disclosure is described with reference to flowcharts and/or block diagrams of methods, devices (systems), and computer program products according to the embodiments of the present disclosure. It should be understood that each flow and/or block in the flowcharts and/or block diagrams, as well as combinations of flows and/or blocks in the flowcharts and/or block diagrams, may be implemented by computer program instructions. These computer program instructions may be provided to a general purpose computer, a special purpose computer, an embedded processor, or a processor of another programmable data processing device to generate a machine, so that an apparatus configured to implement functions specified in one or more flows of the flowcharts and/or one or more blocks of the block diagrams through instructions executed by a computer or a processor of another programmable data processing device is generated.

These computer program instructions may also be stored in a computer-readable memory capable of directing a computer or another programmable data processing device to operate in a specific manner such that the instructions stored in the computer-readable memory produce an article of manufacture including an instruction apparatus, and the instruction apparatus implements functions specified in one or more flows of the flowcharts and/or one or more blocks of the block diagrams.

These computer program instructions may also be loaded onto a computer or another programmable data processing device such that a series of operational acts are executed on the computer or another programmable device to produce computer-implemented processing, such that the instructions executed on the computer or another programmable device provide steps for implementing functions specified in one or more flows of the flowcharts and/or one or more blocks of the block diagrams.

Although preferred embodiments of the present disclosure have been described, those skilled in the art may make additional changes and modifications to these embodiments once basic inventive concepts are known. Therefore, the appended claims are intended to be interpreted to encompass preferred embodiments as well as all changes and modifications falling within the scope of the present disclosure.

Apparently, a person skilled in the art may make various modifications and variations to the present invention without departing from the spirit and scope of the present invention. Thus, if these modifications and variations to the present invention are within the scope of the claims of the present invention and the equivalent techniques thereof, the present invention is intended to comprise these modifications and variations.

1. A detection method for IGK gene rearrangement, comprising:
obtaining paired-end sequencing data of a test sample; the paired-end sequencing data comprising a first-end sequencing sequence and a second-end sequencing sequence;
assembling based on the first-end sequencing sequence and the second-end sequencing sequence to obtain an assembled sequence;
determining a target alignment gene from a gene reference database based on the assembled sequence; wherein the gene reference database comprises an IGKV gene library, an IGKJ gene library, a Kde gene library and a J_C_intron gene library in a germ cell line, and the target alignment gene comprises at least one of a target V gene, a target J gene, a target Kde gene and a target J_C_intron gene;
determining an IGK gene rearrangement result in the assembled sequence based on the target alignment gene.

2. The detection method of claim 1, wherein the first-end sequencing sequence comprises a plurality of first read sequences, and the second-end sequencing sequence comprises a plurality of second read sequences;
assembling based on the first-end sequencing sequence and the second-end sequencing sequence to obtain an assembled sequence comprises:
traversing the first read sequence to determine a first similar read sequence corresponding to the first read sequence; taking a majority voting based on each group of the first read sequence and the first similar read sequence to obtain a first-end corrected sequence; and traversing the second read sequence to determine a second similar read sequence corresponding to the second read sequence; taking a majority voting based on each group of the second read sequence and the second similar read sequence to obtain a second-end corrected sequence;
assembling based on the first-end corrected sequence and the second-end corrected sequence to obtain the assembled sequence.

3. The detection method according to claim 2, wherein
taking a majority voting based on each group of the first read sequence and the first similar read sequence to obtain a first-end corrected sequence comprises:
determining an amount of similarity based on each group of the first read sequence and the first similar read sequence; when the amount of similarity is greater than a set value, taking a majority voting on the bases at each position of the first read sequence and the first similar read sequence to obtain a first corrected read sequence; obtaining the first-end corrected sequence according to all the first corrected read sequences;
taking a majority voting based on each group of the second read sequence and the second similar read sequence to obtain a second-end corrected sequence comprises:
determining an amount of similarity based on each group of the second read sequence and the second similar read sequence; when the amount of similarity is greater than the set value, taking a majority voting on the bases at each position of the second read sequence and the second similar read sequence to obtain a second corrected read sequence; obtaining the second-end corrected sequence according to all the second corrected read sequences.

4. The detection method of claim 3, after obtaining the first-end corrected sequence and the second-end corrected sequence, the detection method further comprises:
trimming an adapter sequence from the first corrected read sequence to obtain a first preprocessed read sequence, and obtaining a first-end preprocessed sequence according to all the first preprocessed read sequences; and trimming an adapter sequence from the second corrected read sequence to obtain a second preprocessed read sequence, and obtaining a second-end preprocessed sequence according to all the second preprocessed read sequences;
assembling based on the first-end corrected sequence and the second-end corrected sequence to obtain the assembled sequence comprises:
assembling based on the first-end preprocessed sequence and the second-end preprocessed sequence to obtain the assembled sequence.

5. The detection method of claim 4, after obtaining the first-end preprocessed sequence and the second-end preprocessed sequence, the detection method further comprising:
deleting the first preprocessed read sequence having a length lower than a first set length to obtain a first-end sequence to be assembled; and deleting the second preprocessed read sequence having a length lower than the first set length to obtain a second-end sequence to be assembled;
assembling based on the first-end preprocessed sequence and the second-end preprocessed sequence to obtain the assembled sequence comprises:
assembling based on the first-end sequence to be assembled and the second-end sequence to be assembled to obtain the assembled sequence.

6. The detection method according to claim 5, wherein a value of the first set length ranges from 10 bp to 100 bp.

7. The detection method of claim 5, wherein assembling based on the first-end sequence to be assembled and the second-end sequence to be assembled to obtain the assembled sequence comprises:
obtaining a reverse complementary read sequence of the second preprocessed read sequence;
determining an overlapping sequence according to the first preprocessed read sequence and the reverse complementary read sequence;
when a length of the overlapping sequence is not lower than a second set length, deleting the overlapping sequence from the reverse complementary read sequence to obtain a read sequence to be assembled;
splicing the first preprocessed read sequence and the read sequence to be assembled to obtain an assembled read sequence;
obtaining the assembled sequence based on all the assembled read sequences.

8. The detection method of claim 1, wherein determining a target alignment gene from a gene reference database based on the assembled sequence comprises:
determining a target alignment gene corresponding to each of the assembled read sequences from the target gene reference database based on set alignment parameters.

9. The detection method according to claim 8, wherein the set alignment parameters comprise: the similarity between alignment fragments in the assembled read sequence and the target alignment gene being not less than 90%, and a length of the alignment fragment ranging from 4 to 11.

10. The detection method according to claim 1, wherein, when the target alignment gene comprises only the target V gene and the target J gene, determining an IGK gene rearrangement result in the assembled sequence based on the target alignment gene comprises:
obtaining a nucleotide position comprising a phenylalanine residue in the target J gene, and determining a termination point in the assembled sequence based on the nucleotide position;
detecting a cysteine residue in the assembled sequence within a set range before the termination point, and taking a position point of the cysteine residue closest to the termination point as a starting point; the set range being assembled sequence fragments from the termination point to 60 bp to 90 bp before the termination point;
determining a CDR3 region in the assembled sequence according to the starting point and the termination point.

11. The detection method according to claim 1, wherein, when the target alignment gene comprise only the target V gene and the target J gene, determining an IGK gene rearrangement result in the assembled sequence based on the target alignment gene comprises:
performing clustering analysis on the assembled sequence based on the target V gene and the target J gene to obtain a quantity of clone sequences and a proportion of clone sequences in the assembled sequence.

12. A detection apparatus for IGK gene rearrangement, comprising:
an acquisition module configured to obtain paired-end sequencing data of a test sample; the paired-end sequencing data comprising a first-end sequencing sequence and a second-end sequencing sequence;
an assembly module configured to assemble based on the first-end sequencing sequence and the second-end sequencing sequence to obtain an assembled sequence;
an alignment module configured to determine a target alignment gene from a gene reference database based on the assembled sequence; wherein the gene reference database comprises an IGKV gene library, an IGKJ gene library, a Kde gene library and a J_C_intron gene library in a germ cell line, and the target alignment gene comprises at least one of a target V gene, a target J gene, a target Kde gene and a target J_C_intron gene;
a determination module configured to determine an IGK gene rearrangement result in the assembled sequence based on the target alignment gene.

13. An electronic device comprising a processor and a memory coupled to the processor, the memory storing instructions that, when executed by the processor, cause the electronic device to perform steps of the detection method of claim 1.

14. A computer-readable storage medium having stored thereon a computer program, wherein when the computer program is executed by a processor, steps of the detection method of claim 1 are implemented.
